(19) United States
(12) Patent Application Publication
(10) Pub. No.: US 2007/0048757 A1
(43) Pub. Date: Mar. 1, 2007

(54) METHODS FOR CHARACTERIZING CELLS USING AMPLIFIED MICRO RNAS

(75) Inventors: Kai Qin Lao, Pleasanton, CA (US); Neil A. Straus, Emeryville, CA (US); Will Bloch, White Salmon, WA (US)

Correspondence Address: MILA KASAN, PATENT DEPT.;APPLIED BIOSYSTEMS, 850 LINCOLN CENTRE DRIVE, FOSTER CITY, CA, 94404, US

(73) Assignee: APPLERA CORPORATION, Foster City, CA (US)

(21) Appl. No.: 11/421,460

(22) Filed: May 31, 2006

Related U.S. Application Data
(60) Provisional application No. 60/686,521, filed on May 31, 2005.
(60) Provisional application No. 60/708,946, filed on Aug. 16, 2005.

(51) Int. Cl. C12Q 1/68; C12P 19/34

(52) U.S. Cl. 435/6; 435/91.2

(57) ABSTRACT

The present teachings provide methods, compositions, and kits for reverse transcribing and amplifying small nucleic acids such as micro RNAs. In some embodiments, the present teachings provide methods of forming micro RNA signatures from single cells, including stem cells. In some embodiments, the present teachings provide methods for determining the identity and/or purity of cells. The present employ performing a multiplexed reverse transcription reaction comprising stem-loop reverse transcription primers, which optionally undergoes temperature cycling, followed by a multiplexed PCR-based pre-amplification reaction, and a subsequently a plurality of lower-plex decoding PCRs.

### CROSS REFERENCE TO RELATED APPLICATIONS

This application claims a priority benefit under 35 U.S.C. § 119(e) from U.S. Patent Application No. 60/686,521, filed May 31, 2005, and to U.S. Patent Application No. 60/708,946, filed Aug. 16, 2005, the contents of which are incorporated herein by reference.

### FIELD

The present teachings are in the field of molecular and cell biology, specifically in the field of defining and characterizing cells using amplified nucleic acids such as micro RNAs.

### INTRODUCTION

Numerous fields in molecular biology require the identification of target polynucleotide sequences. Reverse transcription and amplification are two frequently used procedures employed to query the identity of target polynucleotides. The increasing amount of sequence information available to scientists in the post-genomics era has produced an increased need for rapid, reliable, low-cost, high-throughput, sensitive, and accurate methods to query complex nucleic acid samples. Methods of defining and characterizing cells have been hindered by robust amplification technologies, as well as the molecular complexity of conventionally analyzed molecules such as messenger RNA. Micro RNAs are a recently discovered class of molecules that offer great promise in understanding cell function. However, quantitative analysis of micro RNA has been hindered by their relatively short size. Analysis of cell type in the context of stem cells is a therapeutically important area where advances in micro RNA quantitation are needed.

### SUMMARY

In some embodiments, the present teachings provide a method of forming a micro RNA signature from a small sample, said method comprising; contacting a first target micro RNA with a first stem-loop primer, and a second target micro RNA with a second stem-loop primer; reverse transcribing the first micro RNA and the second micro RNA by extension of the first stem-loop primer and the second stem-loop primer, to form a collection of extension products; performing a PCR-based pre-amplification on the collection of extension products to form a collection of PCR-based pre-amplification products, wherein the collection of PCR-based pre-amplification products comprises a PCR-based pre-amplification first micro RNA product that was amplified with a first PCR-based pre-amplification primer pair, and a PCR-based pre-amplification second micro RNA product that was amplified with a second PCR-based pre-amplification primer pair; dividing the collection of PCR-based pre-amplification products into a first vessel and a second vessel; amplifying the PCR-based pre-amplification first micro RNA product in the first vessel with a first decoding primer pair, and amplifying the PCR-based pre-amplification second micro RNA product in the second vessel with a second decoding primer pair, wherein the first decoding primer pair comprises substantially the same sequence as the first PCR-based pre-amplification primer pair, and wherein the second decoding primer pair comprises substantially the same sequence as the second PCR-based pre-amplification primer pair; detecting amplification of the first micro RNA target amplified in the first vessel and detecting amplification of the second micro RNA target amplified in the second vessel to form a micro RNA signature from the single cell; and, forming a micro RNA signature from the single cell.

In some embodiments, the first decoding primer pair comprises a universal reverse primer encoded by the first stem-loop primer, and a first micro RNA specific forward primer, wherein the first micro RNA-specific forward primer comprises a 3′ target-specific portion and a 5′ tail, and, the second decoding primer pair comprises the universal reverse primer encoded by the second stem-loop primer, and a second micro RNA specific forward primer, wherein the second micro RNA-specific forward primer comprises a 3′ target-specific portion and a 5′ tail, wherein the 5′ tail of the first micro RNA specific forward primer is the same sequence as the 5′ tail of the second micro RNA specific forward primer, wherein the universal reverse primer encoded by the first stem-loop primer is substantially the same sequence as the universal reverse primer encoded by the second stem-loop primer, and wherein the 3′ target specific portion of the first micro RNA specific forward primer is different from the 3′ target specific portion of the second micro RNA specific forward primer.

In some embodiments, the present teachings provide a method of determining the identity of a single putative stem cell, said method comprising; comparing a micro RNA signature for a single putative stem cell to a micro RNA signature from a known single stem cell; and, determining the identity of the putative single stem cell. In some embodiments, the single putative stem cell is an embryonic stem cell, a neuronal stem cell, or a hematopoetic stem cell. In some embodiments, the putative single stem cell is a non-stem cell present in an in vivo stem-cell environment.

In some embodiments, the present teachings provide a method of determining the purity of a population of putative stem cells comprising; providing an aliquot of the population of putative stem cells; forming a micro RNA signature of the aliquot; comparing the micro RNA signature of the aliquot to a micro RNA signature from a known cell; and, determining the purity of the population of the putative stem cells.

### BRIEF DESCRIPTION OF THE DRAWINGS

The skilled artisan will understand that the drawings, described below, are for illustration purposes only. The drawings are not intended to limit the scope of the present teachings in any way.

FIG. 1 depicts certain aspects of various compositions according to some embodiments of the present teachings.

FIG. 2 depicts certain aspects of various compositions according to some embodiments of the present teachings.

FIG. 3 depicts certain sequences of various compositions according to some embodiments of the present teachings.

FIG. 4 depicts one single-plex assay design according to some embodiments of the present teachings.

FIG. 5 depicts an overview of a multiplex assay design according to some embodiments of the present teachings.

FIG. 6 depicts a multiplex assay design according to some embodiments of the present teachings.

FIG. 7 depicts an overview of an assay configuration according to some embodiments of the present teachings.

FIG. 8 depicts one method for reverse transcribing and amplifying a plurality of target polynucleotides according to some embodiments of the present teachings.

FIG. 9 depicts an overview of assessing cell identity in the context of stem cell differentiation.

### DESCRIPTION OF THE EXEMPLARY EMBODIMENTS

Aspects of the present teachings may be further understood in light of the following examples, which should not be construed as limiting the scope of the present teachings in any way. The section headings used herein are for organizational purposes only and are not to be construed as limiting the described subject matter in any way. All literature and similar materials cited in this application, including but not limited to, patents, patent applications, articles, books, treatises, and internet web pages are expressly incorporated by reference in their entirety for any purpose. When definitions of terms in incorporated references appear to differ from the definitions provided in the present teachings, the definition provided in the present teachings shall control. It will be appreciated that there is an implied “about” prior to the temperatures, concentrations, times, etc discussed in the present teachings, such that slight and insubstantial deviations are within the scope of the present teachings herein. In this application, the use of the singular includes the plural unless specifically stated otherwise. For example, “a primer” means that more than one primer can, but need not, be present; for example but without limitation, one or more copies of a particular primer species, as well as one or more versions of a particular primer type, for example but not limited to, a multiplicity of different forward primers. Also, the use of “comprise”, “comprises”, “comprising”, “contain”, “contains”, “containing”, “include”, “includes”, and “including” are not intended to be limiting. It is to be understood that both the foregoing general description and the following detailed description are exemplary and explanatory only and are not restrictive of the invention.

### SOME DEFINITIONS

As used herein, the term “target polynucleotide” refers to a polynucleotide sequence that is sought to be amplified. The target polynucleotide can be obtained from any source, and can comprise any number of different compositional components. For example, the target can be nucleic acid (e.g. DNA or RNA), transfer RNA, siRNA, and can comprise nucleic acid analogs or other nucleic acid mimic, though typically the target will be messenger RNA (mRNA) and/or micro RNA (miRNA). The target can be methylated, non-methylated, or both. The target can be bisulfite-treated and non-

methylated cytosines converted to uracil. Further, it will be appreciated that “target polynucleotide” can refer to the target polynucleotide itself, as well as surrogates *thereof, for example amplification products, and native sequences. In some embodiments, the target polynucleotide is a short DNA molecule derived from a degraded source, such as can be found in for example but not limited to forensics samples (see for example Butler, 2001, Forensic DNA Typing: Biology and Technology Behind STR Markers. The target polynucleotides of the present teachings can be derived from any of a number of sources, including without limitation, viruses, prokaryotes, eukaryotes, for example but not limited to plants, fungi, and animals. These sources may include, but are not limited to, whole blood, a tissue biopsy, lymph, bone marrow, amniotic fluid, hair, skin, semen, biowarfare agents, anal secretions, vaginal secretions, perspiration, saliva, buccal swabs, various environmental samples (for example, agricultural, water, and soil), research samples generally, purified samples generally, cultured cells, and lysed cells. It will be appreciated that target polynucleotides can be isolated from samples using any of a variety of procedures known in the art, for example the Applied Biosystems ABI Prism™ 6100 Nucleic Acid PrepStation, and the ABI Prism™ 6700 Automated Nucleic Acid Workstation, Boom et al., U.S. Pat. No. 5,234,809, mirVana RNA isolation kit (Ambion), etc. It will be appreciated that target polynucleotides can be cut or sheared prior to analysis, including the use of such procedures as mechanical force, sonication, restriction endonuclease cleavage, or any method known in the art. In general, the target polynucleotides of the present teachings will be single stranded, though in some embodiments the target polynucleotide can be double stranded, and a single strand can result from denaturation.

As used herein, the term “small sample” refers to a collection of biomolecules that typically requires amplification in order to detect and quantity the nucleic acids contained therein. For example, a small sample can be collected from a single cell. In some embodiments, a small sample is 2 cells. In some embodiments, a small sample is less than 100 cells. In some embodiments, a small sample is less than 500 cells. In some embodiments, a small sample is less than 1000 cells.

As used herein, the term “extension reaction” refers to an elongation reaction in which the 3′ target specific portion of a primer is extended to form an extension reaction product comprising a strand complementary to the target polynucleotide. In some embodiments, the target polynucleotide is a miRNA molecule and the extension reaction is a reverse transcription reaction comprising a reverse transcriptase, where the 3′ end of a stem-loop primer is extended. In some embodiments, the extension reaction is a reverse transcription reaction comprising a polymerase derived from a Eubacteria. In some embodiments, the extension reaction can comprise rTth polymerase, for example as commercially available from Applied Biosystems catalog number N808-0192, and N808-0098. In some embodiments, the target polynucleotide is a miRNA or other RNA molecule, and the use of polymerases that also comprise reverse transcription properties can allow for a first reverse transcription reaction followed thereafter by an amplification reaction such as a multiplexed pre-amplification PCR in the same reaction vessel, thereby allowing for the consolidation of two reactions in single reaction vessel. In some embodiments, the target polynucleotide is a short DNA-molecule and the extension reaction comprises a polymerase and results in the synthesis of a 2^{nd} strand of DNA.

As used herein, the term “reverse primer” refers to a primer that when extended in a reaction-such as a reverse transcription reaction forms a strand complementary to the target polynucleotide, this strand being referred to as a “first strand”. Following the extension reaction to form the first strand, a forward primer can hybridize to the first strand and be extended to form a second strand. In some embodiments, a stem-loop primer functions as a first reverse primer in a reverse transcription reaction. Thereafter, a second reverse primer that was encoded by the stem-loop primer can be employed. This second reverse primer typically comprises the same sequence as, or substantially the same sequence as, the loop of the stem-loop primer.

As used herein, the term “forward primer” refers to a primer that hybridizes to an extension product, for example an extension product resulting from a reverse transcription reaction, and works to copy a complementary strand in a PCR. The forward primer typically comprises a 3′ target specific portion that hybridizes to the target, as well as a non-complementary 5′ tail.

As used herein, the term “hybridization” refers to the complementary base-pairing interaction of one nucleic acid with another nucleic acid that results in the formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing primers to complementary and substantially complementary target sequences are well known, e.g., as described in Nucleic Acid Hybridization, A Practical Approach, B. Hames and S. Higgins, eds., IRL Press, Washington, D.C. (1985) and J. Wetmur and N. Davidson, Mol. Biol. 31:349 et seq. (1968). In general, whether such annealing takes place is influenced by,!among other things, the length of the polynucleotides and the complementary, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the single stranded nucleic acids of the present teachings. However, if the number of base pair mismatches is so great that no hybridization can occur under minimally stringent conditions then the sequence is generally not a complementary target sequence. Thus, complementarity herein is meant that primers are sufficiently complementary to the target sequence to hybridize under the selected reaction conditions to achieve the ends of the present teachings.

As used herein, when the phrase “substantially the same as” is used to refer to a sequence that is largely complementary to the corresponding sequence. For the avoidance of doubt, “substantially the same as” will typically mean that the sequence is at least 90 percent the same as the corresponding sequence. Said another way, substantially the same as used herein refers to sequence that is at least 90 percent homologous to the corresponding sequence. Further, when referring for example to the fact that the reverse primer comprises substantially the same sequence as the sequence contained in the loop of the stem-loop reverse transcription primer, it will be appreciated that two issue are at play. First, at least 70 percent of the sequence of the loop is included in the reverse primer. Second, of that at least 70 percent, at least 90 percent is the same as (homologous to) the corresponding loop sequence.

As used herein, the term “amplifying” refers to any means by which at least a part of a target polynucleotide and/or target polynucleotide surrogate is reproduced, typically in a template-dependent manner, including without limitation, a broad range of techniques for amplifying nucleic acid sequences, either linearly or exponentially. In some embodiments, amplification can be achieved in a self-contained integrated approach comprising sample preparation and detection, as described for example in U.S. Pat. Nos. 6,153,425 and 6,649,378. Reversibly modified enzymes, for example but not limited to those described in U.S. Pat. No. 5,773,258, are also within the scope of the disclosed teachings. The present teachings also contemplate various uracil-based decontamination strategies, wherein for example uracil can be incorporated into an amplification reaction, and subsequent carry-over products removed with various glycosylase treatments (see for example U.S. Pat. No. 5,536,649, and U.S. Non-Provisional Patent Application 11/173,112 to Andersen et al.,). Those in the art will understand that any protein with the desired enzymatic activity can be used in the disclosed methods and kits. Descriptions of DNA polymerases, including reverse transcriptases, uracil N-glycosylase, and the like, can be found in, among other places, Twyman, Advanced Molecular Biology, BIOS Scientific Publishers, 1999; Enzyme Resource Guide, rev. 092298, Promega, 1998; Sambrook and Russell; Sambrook et al.; Lehninger; PCR: The Basics; and Ausbel et al.

The term “corresponding” as used herein refers to a specific relationship between the elements to which the term refers. Some non-limiting examples of corresponding include: a reverse primer can correspond with a target polynucleotide, and vice versa. A forward primer can correspond with a target polynucleotide, and vice versa.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, the term “reaction vessel” generally refers to any container in which a reaction can occur in accordance with the present teachings. In some embodiments, a reaction vessel can be an eppendorf tube, and other containers of the sort in common practice in modern molecular biology laboratories. In some embodiments, a reaction vessel can be a well in microtitre plate, a spot on a glass slide, or a well in an Applied Biosystems TaqMan Low Density Array for gene expression (formerly MicroCard™) For example, a plurality of reaction vessels can reside on the same support. In some embodiments, lab-on-a-chip like devices, available for example from Caliper and Fluidigm, can provide for reaction vessels. In some embodiments, various microfluidic approaches as described in U.S. Provisional Application 60/545674 to Wenz et al., can be employed. It will be recognized that a variety of reaction vessels are available in the art and can be used in the context of the present teachings.

As used herein, the term “PCR-based pre-amplification” refers to a process wherein a plurality of target specific primers are included in a multiplexed amplification reaction, and the multiplexed amplification reaction, typically a PCR, undergoes a limited number of cycles so that the pre-amplification reaction ends prior to the PCR plateau and/or reagent depletion. The term “PCR-based pre-amplification” can be considered to indicate that a secondary amplification reaction is subsequently performed, typically of lower plexy level than the pre-amplification reaction. This secondary amplification reaction, typically a plurality of secondary amplification reactions, can employ the same target-specific primer pairs employed in the multiplexed pre-amplification reaction. However, each secondary amplification reaction typically comprises a single or a few target-specific primer pairs. Further examples of pre-amplification approaches can be found for example in U.S. Pat. No. 6,605,451 to Xtrana, and U.S. patent application Ser. 10/723,520 to Andersen et al.,

As used herein, the term “detection” refers to any of a variety of ways of determining the presence and/or quantity and/or identity of a target polynucleotide. In some embodiments employing a donor moiety and signal moiety, one may use certain energy-transfer fluorescent dyes. Certain nonlimiting exemplary pairs of donors (donor moieties) and acceptors (signal moieties) are illustrated, e.g., in U.S. Pat. Nos. 5,863,727; 5,800,996; and 5,945,526. Use of some combinations of a donor and an acceptor have been called FRET (Fluorescent Resonance Energy Transfer). In some embodiments, fluorophores that can be used as signaling probes include, but are not limited to, rhodamine, cyanine 3 (Cy 3), cyanine 5 (Cy 5), fluorescein, Vic™, Liz™, Tamra™, 5-Fam™, 6-Fam™, and Texas Red (Molecular Probes). (Vic™, Liz™, Tamra™, 5-Fam™, and 6-Fam™ (all available from Applied Biosystems, Foster City, Calif.). In some embodiments, the amount of detector probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator. According to some embodiments, one can employ an internal standard to quantify the amplification product indicated by the fluorescent signal. See, e.g., U.S. Pat. No. 5,736,333. Devices have been developed that can perform a thermal cycling reaction with compositions containing a fluorescent indicator, emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; and 6,174,670, and include, but are not limited to the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), and the ABI GeneAmp® 7500 Sequence Detection System (Applied Biosystems). In some embodiments, each of these functions can be performed by separate devices. For example, if one employs a Q-beta replicase reaction for amplification, the reaction may not take place in a thermal cycler, but could include a light beam emitted at a specific wavelength, detection of the fluorescent signal, and calculation and display of the amount of amplification product. In some embodiments, combined thermal cycling and fluorescence detecting devices can be used for precise quantification of target nucleic acid sequences in samples. In some embodiments, fluorescent signals can be detected and displayed during and/or after one or more thermal cycles, thus permitting monitoring of amplification products as the reactions occur in “real time.” In some embodiments, one can use the amount of amplification product and number of amplification cycles to calculate how much of the target nucleic acid sequence was in the sample prior to amplification. In some embodiments, one could simply monitor the amount of amplification product after a predetermined number of cycles sufficient to indicate the presence of the target nucleic acid sequence in the sample. One skilled in the art can easily determine, for any given sample type, primer sequence, and reaction condition, how many cycles are sufficient to determine the presence of a given target polynucleotide. As used herein, determining the presence of a target can comprise identifying it, as well as optionally quantifying it. In some embodiments, the amplification products can be scored as positive or negative as soon as a given number of cycles is complete. In some embodiments, the results may be transmitted electronically directly to a database and tabulated. Thus, in some embodiments, large numbers of samples can be processed and analyzed with less time and labor when such an instrument is used. In some embodiments, different detector probes may distinguish between different target polynucleoteides. A non-limiting example of such a probe is a 5′-nuclease fluorescent probe, such as a TaqMan® probe molecule, wherein a fluorescent molecule is attached to a fluorescence-quenching molecule through an oligonucleotide link element. In some-embodiments, the oligonucleotide link element of the 5′-nuclease fluorescent probe binds to a specific sequence of an identifying portion or its complement. In some embodiments, different 5′-nuclease fluorescent probes, each fluorescing at different wavelengths, can distinguish between different amplification products within the same amplification reaction. For example, in some embodiments, one could use two different 5′-nuclease fluorescent probes that fluoresce at two different wavelengths (WL_{A} and WL_{B}) and that are specific to two different regions of two different extension reaction products (A′ and B′, respectively). Amplification product A′ is formed if target polynucleotide A is in the sample, and amplification product B′ is formed if target polynucleotide B is in the sample. In some embodiments, amplification product A′ and/or B′ may form even if the appropriate target polynucleotide is not in the sample, but such occurs to a measurably lesser extent than when the appropriate target polynucleotide is in the sample. After amplification, one can determine which specific target nucleic acid sequences are present in the sample based on the wavelength of signal detected and their intensity. Thus, if an appropriate detectable signal value of only wavelength WL_{A} is detected, one would know that the sample includes target polynucleotide A, but not target polynucleotide B. If an appropriate detectable signal value of both wavelengths WL_{A} and WL_{B} are detected, one would know that the sample includes both target polynucleotide A and target polynucleotide B. In some embodiments, detection can be achieved by various microarrays and related software such as the Applied Biosystems. Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003). It will also be appreciated that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. Detection of unlabeled reaction products, for example using mass spectrometry, is also within the scope of the current teachings.

### Exemplary Embodiments

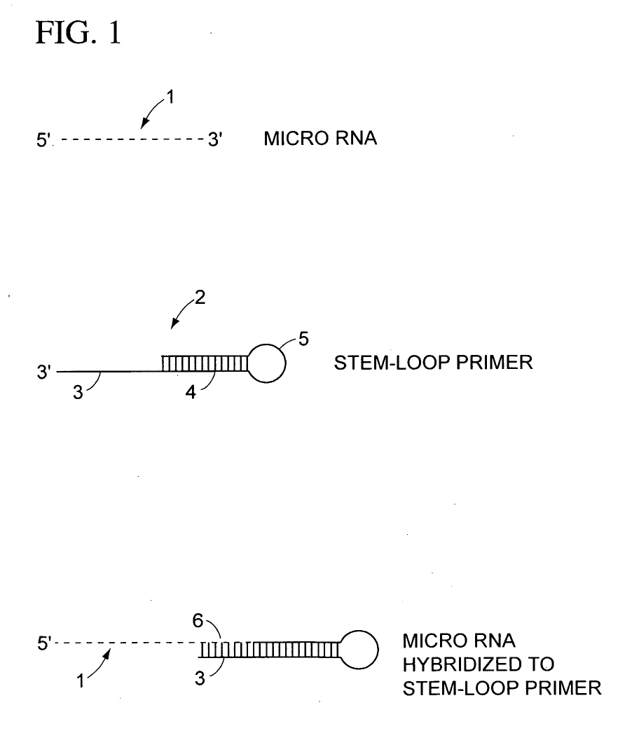

FIG. 1 depicts certain compositions according to some embodiments of the present teachings. Top, a miRNA molecule (1, dashed line) is depicted. Middle, a stem-loop primer (2) is depicted, illustrating a 3′ target specific portion (3), a stem (4), and a loop (5). Bottom, a miRNA hybridized to a stem-loop primer is depicted, illustrating the 3′ target specific portion of the stem-loop primer (3) hybridized to the 3′ end region of the miRNA (6).

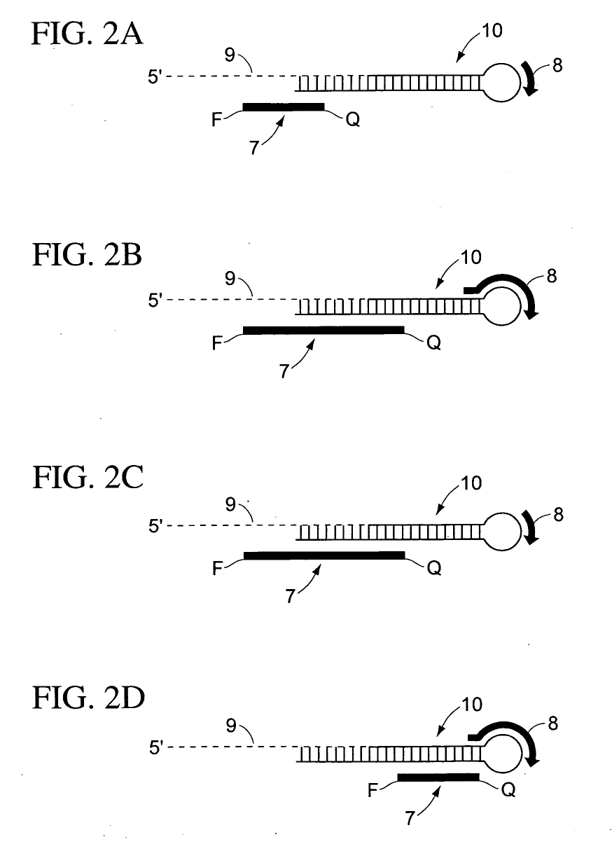

As shown in FIG. 2, a target polynucleotide (9, dotted line) is illustrated to show the relationship with various components of the stem-loop primer (10), the detector probe (7), and the reverse primer (8), according to various non-limiting embodiments of the present teachings. For example as shown in FIG. 2A, in some embodiments the detector probe (7) can correspond with the 3′ end region of the target polynucleotide in the amplification product as well as a region upstream from the 3′ end region of the target polynucleotide in the amplification product. (Here, the detector probe is depicted as rectangle (7) with an F and a Q, symbolizing a TaqMan 5′ nuclease-cleavable probe with a florophore (F) and a quencher (Q)). Also shown in FIG. 2A, the loop can correspond to the reverse primer (8). In some embodiments as shown in FIG. 2B, the detector probe (7) can correspond with a region of the amplification product corresponding with the 3′ end region of the target polynucleotide in the amplification product, as well as a region upstream from the 3′ end region of the target polynucleotide in the amplification product, as well as the stem-loop primer stem in the amplification product. Also shown in FIG. 2B, the upstream region of the stem, as well as the loop, can correspond to the reverse primer (8). In some embodiments as shown in FIG. 2C, the detector probe can correspond to the amplification product in a manner similar to that shown in FIG. 2B, but the loop can correspond to the reverse primer (8). In some embodiments as shown in FIG. 2D, the detector probe (7) can correspond with the stem-loop primer stem in the amplification product. Also shown in FIG. 2D, the upstream region of the stem, as well as the loop can correspond to the reverse primer (8). It will be appreciated that various related strategies for implementing the different functional regions of these compositions are possible in light of the present teachings, and that such derivations are routine to one having ordinary skill in the art without undue experimentation.

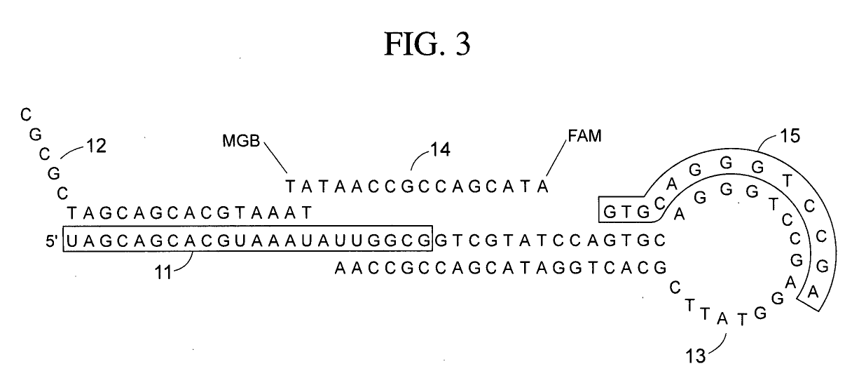

FIG. 3 depicts the nucleotide relationship for the micro RNA MiR-16 (boxed, 11) according to some embodiments of the present teachings. Shown here is the interrelationship of MiR-16 to a forward primer (12), a stem-loop primer (13), a TaqMan detector probe (14), and a reverse primer (boxed, 15). The TaqMan probe comprises a 3′ minor groove binder (MGB), and a 5′ FAM florophore. It will be appreciated that in some embodiments of the present teachings the detector probes, such as for example TaqMan 5′ nuclease-cleavable probes, can hybridize to either strand of an amplification product. For example, in some embodiments the detector probe can hybridize to the strand of the amplification product corresponding to the first strand synthesized. In some embodiments, the detector probe can hybridize to the strand of the amplification product corresponding to the second strand synthesized.

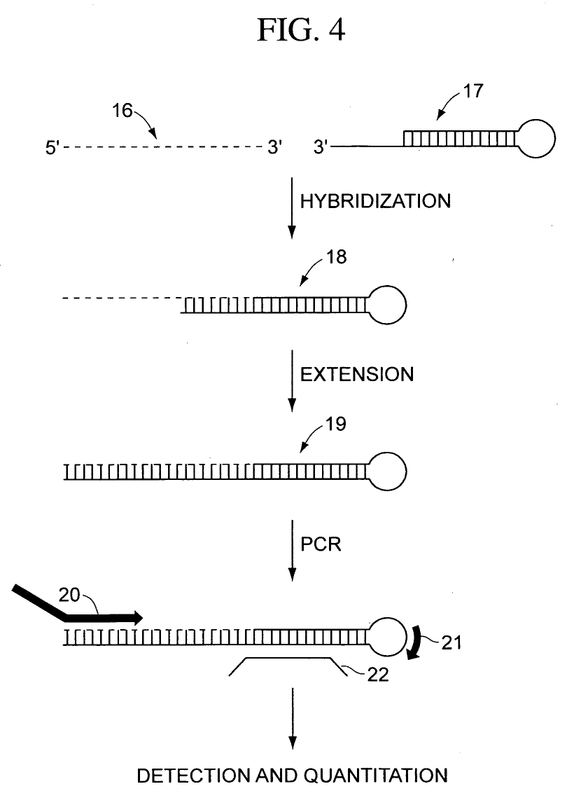

FIG. 4 depicts a single-plex assay design according to some embodiments of the present teachings. Here, a miRNA molecule (16) and a stem-loop primer (17) are hybridized together (18). The 3′ end of the stem-loop primer of the target:stem-loop primer composition is extended to form an extension product (19) that can be amplified in a PCR. The PCR can comprise a miRNA specific forward primer (20) and a reverse primer (21). The detection of a detector probe (22) during the amplification allows for quantitation of the miRNA.

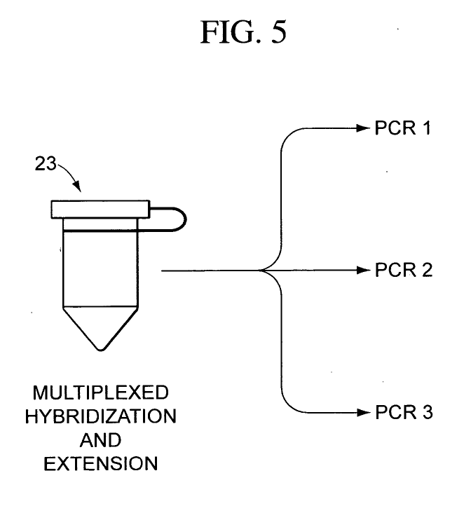

FIG. 5 depicts an overview of a multiplex assay design according to some embodiments of the present teachings. Here, a multiplexed hybridization and extension reaction is performed in a first reaction vessel (23). Thereafter, aliquots of the extension reaction products from the first reaction vessel are transferred into a plurality of amplification reactions (here, depicted as PCRs 1, 2, and 3) in a plurality of second reaction vessels. Each PCR can comprise a distinct primer pair and a distinct detector probe. In some embodiments, a distinct primer pair but the same detector probe can be present in each of a plurality of PCRs.

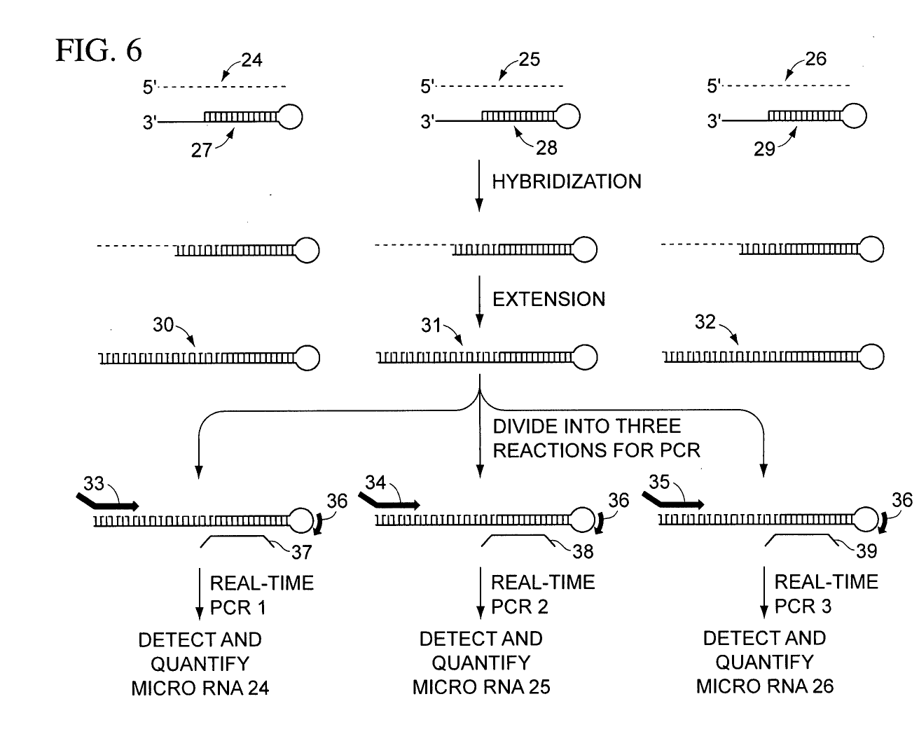

FIG. 6 depicts a multiplex assay design according to some embodiments of the present teachings. Here, three different miRNAs (24, 25, and 26) are queried in a single hybridization reaction mixture comprising three different stem-loop primers (27, 28, and 29). Following hybridization and extension to form extension products (30, 31, and 32), the extension products are divided into three separate amplification reaction mixtures. (Though not explicitly shown, it will be appreciated that a number of copies of the molecules depicted by 30, 31, and 32 can be present, such that each of the three amplification reactions can have copies of 30, 31, and 32.) PCR 1 comprises a forward primer specific for miRNA 24 (33), PCR 2 comprises a forward primer specific for miRNA 25 (34), and PCR 3 comprises a forward primer specific for miRNA 26 (35). Each of the forward primers can further comprise a non-complementary tail portion. PCR 1, PCR 2, and PCR 3 all comprise the same universal reverse primer 36. Further, PCR 1 comprises a distinct detector probe (37) that corresponds to the 3′ end region of miRNA 24 and the stem of stem-loop primer 27, PCR 2 comprises a distinct detector probe (38) that corresponds to the 3′ end region of miRNA 25 and the stem of stem-loop primer 28, and PCR 3 comprises a distinct detector probe (39) that corresponds to the 3′ region of miRNA 26 and the stem of stem-loop primer 29.

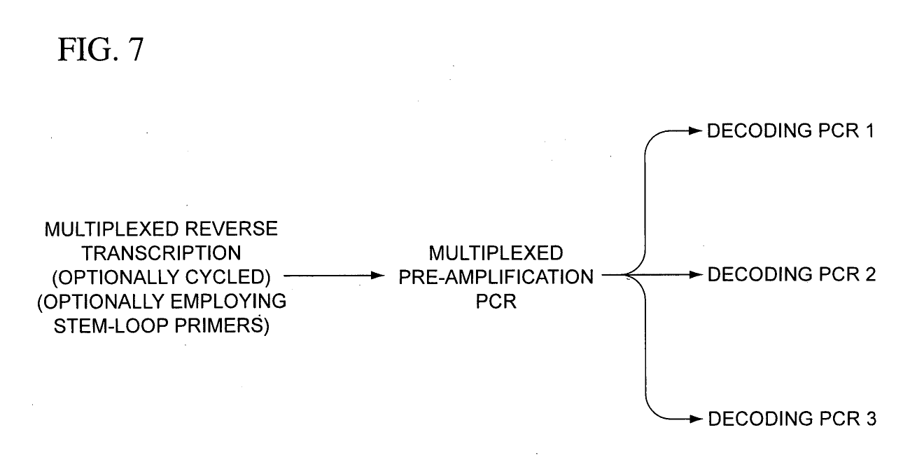

FIG. 7 illustrates an overview of a process according to some embodiments of the present teachings. Here, a multiplexed reverse transcription reaction is performed on a plurality of nucleic acids, such as micro RNAs. This reverse transcription can be cycled according to some embodiments of the present teachings, for example in a 2 segment cycling procedure, or a-three segment cycling procedure. Also, this reverse transcription reaction can comprise stem-loop primers. Following the reverse transcription reaction, and optionally occurring in the same vessel as the reverse transcription reaction, a multiplexed pre-amplification PCR can be performed. This multiplexed pre-amplification PCR can then be followed by a plurality of separate decoding PCRs, here a PCR 1, a PCR 2, and a PCR 3. Pre-amplification of target polynucleotides (see for example U.S. patent application Ser. No. 10/723,520 to Andersen et al., and U.S. Pat. No. 6,605,451 to Xtrana) can pre-amplify cDNA more than 1,000-fold for up to kilo-plexed genes. Subsequent lower-plex ampification reactions can then decode these multiplexed reactions, thereby allowing for detection and quantitation of a plurality of different target polynucleotides with an economy of reagents. The identify and quantity of one, some, or all, of the micro RNAs in a given cell type, or collection of cell types, can be referred to as a ‘micro RNA signature’, and can be achieved by application of the methods of the present teachings. Discussion of signatures can be found, for example, in U.S. Pat. No. 6,110,711 and U.S. Pat. No. 5,514,545, wherein messenger RNA signatures are discussed in such contexts as microarrays. The methods of the present teachings allow for defining micro RNA signatures from individual cells, such as individual stem cells and cells arising therefrom. The present teachings provide for high multiplexing, including up to 220-plex and beyond, and for the derivation of signatures representing large numbers of target micro RNAs from single cells.

Cycling the Reverse Transcription (RT) Reaction

In some embodiments, the present teachings provide methods for enhanced reverse transcription primer utilization by thermal cycling. Without intending to be mechanistically limiting, the cycling methods of the present teachings are believed to disrupt errant primer-low temperature associations and permit repeated-homologous primer/RNA nucleations, thus increasing the amount of reverse transcription reaction, product. The methods of the present teachings can be applied in a number of contexts, including increasing the reverse transcription products in miRNA reverse transcription reactions as well as increasing the reverse transcription products in multiplexed RT-PCR reactions comprising messenger RNA (mRNA) as target polynucleotides.

Conventional RT reactions are performed at a single relatively low temperature, 37-42° C. for a long period of time (30 minutes to 1 hour, see for example Sambrook et al., 3^{rd} Edition). Typically, the aim of these reactions has been the production of long cDNA, using for example poly (T) and random primers to convert mRNA into cDNA. The long time intervals of incubation allow the RT reaction to go to completion, hopefully producing the longest cDNA molecules possible. A couple of relevant considerations provided by the present teachings include: (1) If the RNA targets are short or the defined cDNA product is short, long incubations times are not necessary for polymerase to reverse transcribe the template. (2) Single low hybridization temperatures permit non-homologous associations and intra-strand collapse to interfere with bona fide priming activity.

The kinetic principles behind homologous low temperature primer hybridization are complex and poorly defined. At these low temperatures short complementary regions can form stable intra-molecular and inter-molecular associations that interfere with bona fide complementary hybridizations. Because of the large difference in concentration:between the primers and the target RNA's typically present in an RT reaction, only a small fraction of the primers anneal productively to the target sequences in a one step RT-reaction. Thus, it was hypothesized that conditions that (1) dissociate these primers from off target associations, (2) dissociate short intra-molecular hybridizations, and (3) permit the re-hybridization of RT primers, can allow excess un-reacted primers another chance to react with RNA target for another round of RT. Further, it was hypothesized that if these conditions do not inactivate reverse transcriptase, then repetition of these conditions can increase the synthesis of cDNA until available target RNA molecules have been exhausted.

While in principle, increasing the primer concentrations from 50-100 nM to 1 uM or higher would be predicted to increase the number of productive primer target hybridizations, such conditions also increase problems of primer to primer interactions particularly in multiplex reactions. It was hypothesized that procedures that recycle original un-reacted primers would increase the efficiency of primer utilization and hence increase the amount of real product in the RT reaction. Given that some RT enzymes can be stable up to 50° C., 60° or higher, a temperature cycling RT reaction scheme was designed. Example 1 in Table 1 illustrates one such cycling reaction design.

Thus, the present teachings contemplate a method for reverse transcribing and amplifying a target polynucleotide comprising: forming a reaction composition comprising the target polynucleotide, a target-specific primer pair, and a reverse transcriptase, wherein the target-specific primer pair comprises a forward primer and a reverse primer; annealing the reverse primer with the target polynucleotide; performing an extension reaction at a first temperature wherein the reverse primer is extended to form an extension product, wherein a subset of the reverse primers are not extended and do not form extension products; raising the reaction to a second temperature, wherein the extension product is substantially non-denatured at the second temperature, wherein the subset of reverse primers that were not extended and did not form extension products are substantially denatured at the second temperature; cycling between the first temperature and the second temperature for at least two cycles; and, amplifying the target polynucleotide in a first amplification reaction, wherein the amplification reaction comprises a PCR using the target-specific primer pair to form a first amplified sample.

In some embodiments, a plurality of different target polynucleotides and a plurality of different target specific primer pairs are employed in a cycled reverse transcription reaction, followed by a multiplexed pre-amplification reaction in the same reaction vessel. In some embodiments, the multiplexed pre-amplification reaction can occur in a different reaction vessel from the reverse transcription reaction.

Cycling in 2 Segments

In some embodiments, the cycling reverse transcription reaction is performed in two segments.

Thus, in some embodiments, the first temperature (a denaturation temperature) is 47 C-53 C, and the second temperature (an annealing/extension temperature) is 37 C-43 C.

In some embodiments, the cycling reverse transcription reaction comprises at least 30 cycles of 1-5 seconds at the first temperature and 45-75 seconds at the second temperature.

In some embodiments, the cycling comprises at least 60 cycles of 1-5 seconds at the first temperature and 25-35 seconds at the second temperature.

Cycling in 3 Segments

In some embodiments, the cycling comprises three segments: a low temperature segment, an intermediate temperature segment, and a high temperature segment. Without intending to be limiting, the predominating reactions occurring during each of the three segments can be considered as follows: the low temperature segment can be considered an annealing segment, the intermediate temperature segment can be considered an extension segment, and the high temperature segment can be considered a denaturation segment. Thus, in some embodiments, the cycling reverse transcription reaction can comprise an initial 16 C for 30 minute incubation, followed by 60 cycles of 20 C for 30 sec, 42 C for 30 sec, and 50 C for 1 second. These 60 cycles can be followed by a step to inactivate the reverse transcriptase, for example by elevating the temperature to 85 C for 5 minutes.

In some embodiments, there can be between 50-70 cycles. In some embodiments, there can be 40-80 cycles. In some embodiments, there can be 30-100 cycles. In some embodiments, there can be greater than 100 cycles.

In some embodiments, the low temperature segment can be at l8-22 C. In some embodiments, the low temperature segment can be 19-21 C. In some embodiments, the low temperature segment can be 15-25 C.

In some embodiments, the low temperature segment can last for 25-35 seconds. In some embodiments, the low temperature segment can last for 20-40 seconds. In some embodiments, the low temperature segment can last 15-60 seconds. In some embodiments, the low temperature segment can last longer than 60 seconds, though it will be appreciated that longer times may add-unnecessary delay-to the acquisition of results.

In some embodiments, the intermediate temperature segment can be 37 C-45 C. In some embodiments, the intermediate temperature segment can be 39 C-43 C.

In some embodiments, the intermediate temperature segment can last for 25-35 seconds. In some embodiments, the intermediate temperature segment can last for 20-40 seconds. In some embodiments, the intermediate temperature segment can last 15-60 seconds. In some embodiments, the intermediate temperature segment can last longer than 60 seconds, though it will be appreciated that longer times may add unnecessary delay to the acquisition of results.

In some embodiments, the high temperature segment can be 48-55 C. In some embodiments, the high temperature segment can be 49-51 C. In some embodiments, the high temperature segment can be higher than 55 C, though it will be appreciated that higher temperatures can denature and/or destroy enzymatic activity.

In some embodiments, the high temperature segment can last 1-10 seconds. In some embodiments, the high temperature segment can last 2-8 seconds. In some embodiments, the high temperature segment can last 1-5 seconds. In some embodiments, the high temperature segment can last longer than 10 seconds, though it will be appreciated that longer times at the high temperature can denature and/or destroy enzymatic activity, especially when longer times are employed. It will further be appreciated that longer times may add unnecessary delay to the acquisition of results.

In some embodiments, especially those in which longer nucleic acids such as messenger RNAs are queried, the target-specific primer pair queries a region of the target polynucleotide that is between 100-150 nucleotides in length. As longer regions are queried, generally, incubation times can be increased, and denaturation temperatures can be increased as well.

In some embodiments, the present teachings provide a reaction composition comprising; a plurality of target-specific primer pairs, wherein the plurality of target-specific primer pairs comprise a forward primer and reverse primer, and, wherein the plurality of reverse primers are capable of reverse transcribing a plurality of target polynucleotides in a cycling reverse transcription reaction; a reverse transcriptase; and, a polymerase. In some embodiments, the plurality of target-specific primer pairs query a region of the plurality of target polynucleotides that is between 100-150 nucleotides in length.

Multiplexed PCR-Based Pre-Amplification Reaction Followed by Lower-Plex PCR Decoding Reactions

In some embodiments, the present teachings further provide multiplexed RT-PCR approaches for defining micro RNA signatures of individual cells. In some embodiments, these approaches comprise a multiplexed RT, which can be cycled as described above, followed by a multiplexed PCR-based pre-amplification, optionally occurring in the same reaction vessel as the RT. The resulting multiplexed PCR-based pre-amplification products can be divided into a plurality of downstream reaction vessels, and each of these downstream reaction vessels can amplify one, or a small number, of the products resulting from the PCR-based pre-amplification. By the intelligent choice of primers, and detector probes, specific targets can be quantified in these downstream amplification reactions, thereby allowing for the determination-of a micro RNA signature for a single cell.

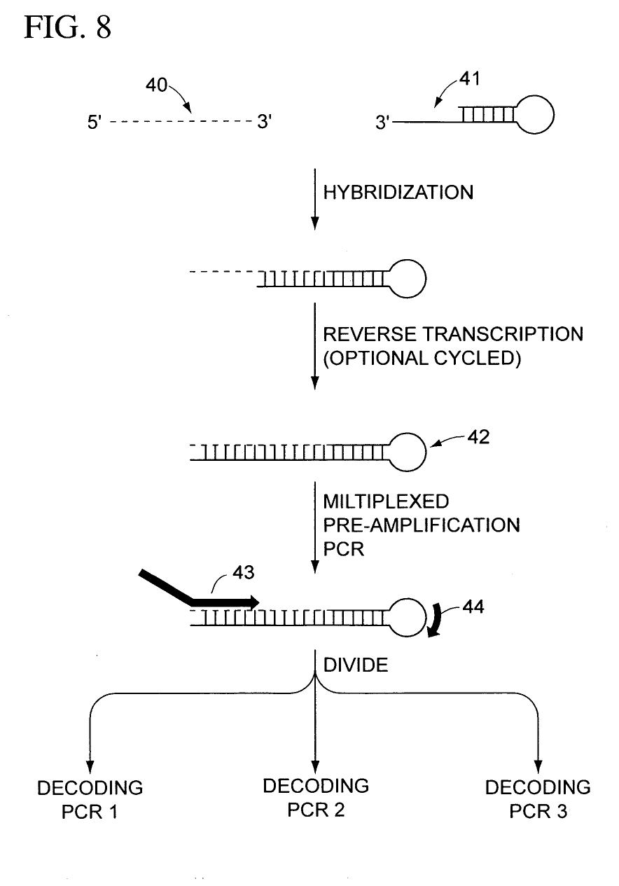

An illustrative reaction overview is depicted in FIG. 8, showing a multiplex assay design according to some embodiments of the present teachings. Here, a miRNA is shown queried in a hybridization reaction comprising a stem-loop primer (41). Following hybridization, a reverse transcription extension reaction can be performed, optionally in a cycling procedure to form extension products (42). The extension products can then undergo a multiplexed pre-amplification PCR employing a micro-RNA specific forward primer (43) and micro-RNA specific reverse primer (44). Thereafter, the products of the multiplexed pre-amplification PCR are divided into three separate decoding amplification reactions (here, PCR 1, PCR 2, and PCR 3).

Though not explicitly shown in FIG. 8, it will be appreciated that in a manner analogous to FIG. 6, a plurality of micro RNA species can exist in this assay, wherein a plurality of target-specific stem-loop primers can be employed in the reverse transcription reaction. Further, the plurality of reverse transcription reaction products can be amplified in the multiplexed pre-amplification PCR employing a plurality of target-specific primer pairs. PCR 1 can comprise a forward primer specific for a first miRNA, PCR 2 can comprise a forward primer specific for a second miRNA, and PCR 3 can comprises a forward primer specific for a third miRNA. Each of the forward primers can further comprise a non-complementary tail portion. PCR 1, PCR 2, and PCR 3 can all comprise the same universal reverse primer that was encoded by a common region of the three different stem-loop primers in the reverse transcription reaction. Further, PCR 1 can comprise a distinct detector probe that corresponds to the 3′ end region of the first miRNA and the stem of a first stem-loop primer, PCR 2 can comprise a distinct detector probe that corresponds to the 3′ end region of the second miRNA and the stem of a second stem-loop primer, and PCR 3 can comprise a distinct detector probe that corresponds to the 3′ region of the third miRNA and the stem of a third stem-loop primer.

Thus, in some embodiments the present teachings contemplate encoding and decoding reaction schemes, wherein a first encoding extension reaction and pre-amplification reaction is followed by a second decoding amplification reaction, as described for example U.S. patent applications Ser. Nos. 11/090,468, 11/090830, U.S. Pat. No. 6,605,451 to Xtrana, and U.S. patent application Ser. No. 10/723,520 to Andersen et al.

In some embodiments, the present teachings contemplate a variety of strategies to minimize the number of different molecules in multiplexed amplification strategies, as described for example in Whitcombe et al., U.S. Pat. No. 6,270,967, U.S. patent application Ser. No. 10/666,806 to Aydin, and U.S. patent application Ser. No. 10/665,671 to Andersen et al.

Stem Cells

In some embodiments, the present teachings can be applied in the context of stem cell identification. For example, a tissue of interest can be a therapeutically desirable stem cell, for example an embryonic stem cell. An aliquot can be collected from a population of in vitro cultured stem cells, and one or many target micro RNA sequences known to be expressed in a known therapeutically desirable stem cell can be compared to the quantitated micro RNA signature of the aliquot according to the assays of the present teachings in order to infer, for example, the level of purity of the population of cultured stem cells. In some embodiments, target micro RNA sequences expressed in the inappropriately differentiated cells can also be quantitated according to the assays of the present teachings. Such inappropriately differentiated cells in this context could comprise, for example, therapeutically undesirable stem cells. It will be appreciated by one of skill in the art that chemically treated stem cell populations in vitro can vary unpredictably in their differentiation; the present teachings provide a rapid and sensitive assay for assessing the status of an aliquot of cells derived from such an in vitro stem cell culture. Generally, by comparing the micro RNA signature of such an aliquot, as determined by the assay methods of the present teachings, to the micro RNA signature present in a therapeutically desirable cell type or therapeutically undesirable cell type, the clinical experimentalist can perform a ‘positive selection’ to keep the therapeutically desirable stem cells and/or a ‘negative selection’ and get rid of undesired cells.

For example, the present teachings contemplate embodiments in which the micro RNA signature of an aliquot of cells from a population is compared to a micro RNA signature known to be present in a therapeutically desirable stem cell, and because of the positive correlation between the micro RNAs signature in the aliquot as compared to the micro RNA signature for the therapeutically desirable stem cell, the experimentalist performs a positive selection and keeps the population of cells.

The present teachings also contemplate embodiments in which the micro RNA signature of an aliquot of cells from a population is compared to a micro RNA signature known to be present in a therapeutically desirable stem cell, and because of the lack of a positive correlation between the micro RNA signature in the aliquot as compared to the micro RNA signature for the therapeutically desirable stem cell, the experimentalist performs a negative selection and discards the population of cells.

The present teachings also contemplate embodiments in which the micro RNA signature of an aliquot of cells from a population is compared to a micro RNA signature present in a therapeutically undesirable cell type (for example an inappropriately differentiated cell type), and because of the positive correlation between the micro RNA signature quantified in the aliquot as compared to the micro RNA signature in the therapeutically undesirable cell type, the experimentalist performs a negative selection and discards the population of cells.

The present teachings also contemplate embodiments in which the micro RNA signature of an aliquot of cells from a population is compared to a micro RNA signature present in a therapeutically undesirable cell type (for example an inappropriately differentiated cell type), and because of the lack of a positive correlation between the micro RNA signature quantified in the aliquot as compared to the micro RNA signature in the therapeutically undesirable cell type, the experimentalist performs a positive selection and keeps the population of cells.

Of course, various combinations of these procedures and analogously related approaches can be employed, as will be appreciated by one of ordinary skill in the art upon consideration of the present teachings

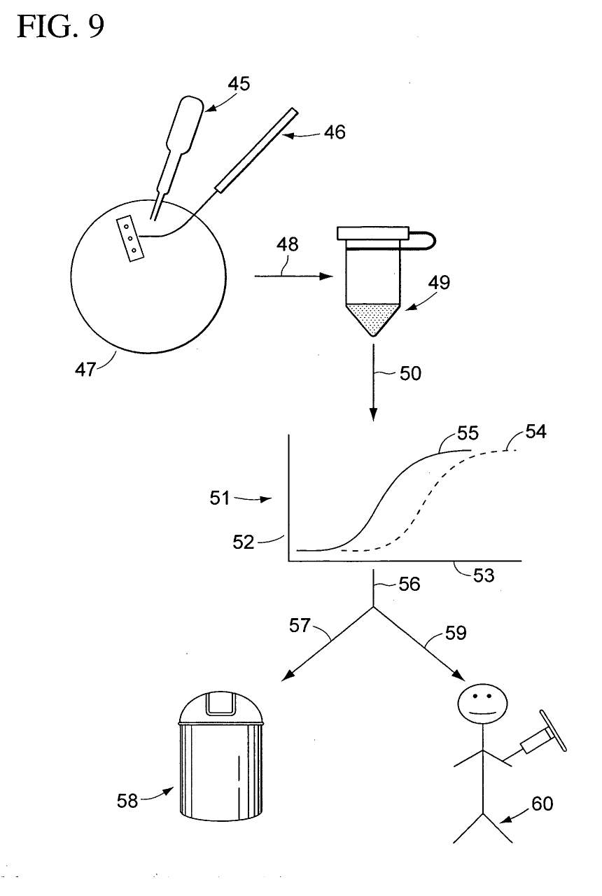

For example, FIG. 9 depicts some illustrative embodiments-of how the methods, of the present teachings can be applied in the context of identifying stem cells. Here, a test sample comprising stem cells in a pipette (45) is collected using a cell scraper (46) from a population of cultured stem cells in a Petri dish (47). After an appropriate sample preparation (48) such as heat lysing, or the use of commercially available Applied Biosystems Tempus Tube™, the prepared test sample (49, shown here in an eppendorf tube) comprising micro RNAs can be analyzed (50) according to the methods of the present teachings, for example in an Applied Biosystems real time PCR assay employing micro RNA specific stem-loop primers and TaqMan™ detector probes, along with an internal reference small RNA and corresponding reagents. The resulting graph (51) indicates signal on the Y axis (52) and cycle number on the X axis (53). The dotted curve (54) represents a reference level and the solid curve (55) represents the target micro RNA sequence. The left-shifted solid curve can indicate a higher expression level of the target micro RNA sequence than normal, and hence provides the diagnosing a biological condition (56). In the context of stem cell identification here, the diagnosing a biological condition can indicate that the population of stem cells in the Petri dish contains undesirable background tissue, and negative selection employed (57), wherein the cells in the Petri dish can be discarded (58). Alternatively, the diagnosing a biological condition can indicate that the population of stem cells in the Petri dish comprises differentiated stem cells of interest and positive selection employed (59), wherein the cells in the Petri dish are collected and injected into a patient (60). Additional teachings for manipulating stem cells can be found in Handbook of Stem Cells, Volume 1 and 2, 2005, Elsevier, edited by Robert Lanza.

In some embodiments of the present teachings, the stem cells can be differentiated into dopaminergic neurons, and after identification of their purity according to the present teachings, injected into the substantial nigra region of a patient suffering from Parkinson's Disease. As another example, the stem cells can be differentiated into cholinergic neurons, and after identification of their purity according to the present teachings, injected into the basal forebrain region of a patient suffering from Alzheimer's Disease. As another example, the stem cells can be differentiated into multipolar choninergic motoneurons, and after identification of their purity according to the present teachings, injected into the ventral spinal cord region of a patient suffering from a spinal cord injury. It will be appreciated that the present teachings, in providing for sensitive and accurate quantification of micro RNAs and the corresponding delineation of cell types, enable a diverse suite of potential clinical applications, as well as basic research applications (see for example Suh et al., Development Biology (2004) 270: 488-498 and Houbaviy et al., Developmental Cell (2003) 5: 351-358.

While the present teachings have been described in terms of these exemplary embodiments, the skilled artisan will readily understand that numerous variations and modifications of these exemplary embodiments are possible without undue experimentation. All such variations and modifications are within the scope of the current teachings. Aspects of the present teachings may be further understood in light of the following examples, which should not be construed as limiting the scope of the teachings in any way.

### EXAMPLE 1

A protocol and reagents than can be used according to some embodiments of the present-teachings is shown in Table 1, proceeding from top to bottom in chronological order, (occasionally showing zeros were [reagent] is not applicable). Use of this method resulted in appropriately lower Ct values in a TaqMan® assay for miR-16 from a single stem cell, as compared to Ct values in a TaqMan® assay for miR-16 from two stem cells. The stem-loop reverse reverse transcription primer, forward primer, reverse primer, TaqMan® probe, that can be used to query miR-1 6 are:

Stem-Loop Reverse Transcription Primer

SEQ ID NO: 1

5′CTCAACTGGTGTCGTGGAGTCGGCAATTCAGTTGAGCGCCATA3′
Forward Primer

SEQ ID NO: 2

5′ACACTCCAGCTGGGTAGCAGCACGTAATA3′
TaqMan Probe

SEQ ID NO: 3

5′6-Fam-TTCAGTTGAGCCGCCAATA-MGB3′

TABLE 1

| Reagent | Volume (ul) | [Stock] | [Final] | | |
|---|---|---|---|---|---|
| STEP1 RT | | | | | 3× Mix |
| 10× Applied Bio-systems | 0.5 | 10 | 1 | | 1.5 |
| cDNA Archiving Kit buffer | | | | | |
| MMLV Reverse Transcriptase | .335 | 50 | 3.35 | (3.3 units/ul) | 1.005 |
| 50 units/ul | | | | | |
| 100 mM dNTP | 0.25 | 100 | 5 | (100 mM/ul) | 0.75 |
| Applied Biosystems | 0.065 | 20 | 0.26 | (0.26 units/ul) | 0.195 |
| RNase Inhibitor | | | | | |
| 192-plex micro RNA stem- | 0.5 | 50 | 5 | (5 nM) | 1.5 |
| loop primer primers (50 mM) | | | | | |
| Total RNA 10 ng/ul samples | 3 | 10 | 6 | | 9 |
| dH20 | 0.35 | 0 | 0 | | 1.05 |
| total Volume | 5 | | | | 15 |
| 16 C. 30 min, | | | | | |
| (42 C. 1 min-50 C. 1 sec) × | | | | | |
| 60 cycles, 85 C. 5 minutes | | | | | |
| STEP 2 PCR-based | | | | | |
| pre-amplification | | | | | |
| 2× universal master mix | 12.5 | 2 | 1 | | 37.5 |
| (Applied Biosystems, No Ung) | | | | | |
| RT-template | 5 | 0 | 0 | | 15 |
| 192-plex new forward | 2.5 | 500 | 50 | (50 uM) | 7.5 |
| primer (500 nM) | | | | | |
| Universal Reverse | 1.25 | 100 | 5 | (5 uM) | 3.75 |
| Primer 100 uM | | | | | |
| AmpiTaq Gold (Applied | 1.25 | 5 | 0.25 | 0.25 units/ul | 3.75 |
| Biosystems) 5 units/ul | | | | | |
| dNTP 100 mM | 0.5 | 100 | 2 | (2 mM) | 1.5 |
| MgCl2 100 mM | 0.5 | 100 | 2 | (2 mM) | 1.5 |
| dH20 | 1.5 | 0 | 0 | | 4.5 |
| total Volume | 25 | | | | 75 |
| 95 C. 10 min, 55 C. 2 min, | | | | | |
| (95 C. 1 sec, 65 C. 1 min) × | | | | | |
| 14 cycles | | | | | |
| 25 ul pre amplification | | | | | |
| PCR template + 75 ul | | | | | |
| dH20 = 100 ul | | | | | |
| STEP 3-Plurality of | | | | | 210× Mix |
| Decoding PCRs (TaqMans ®) | | | | | |
| 2× universal master mix | 5 | 2 | 1 | | 1050 |
| (Applied Biosytems, No Ung) | | | | | |
| Forward Primer 5 uM + | 2 | 10 | 2 | 1 uM FP, 200 nM | 420 |
| TaqMan ® probe (1 uM) | | | | TaqMan ® Probe | |
| Universal Reverse Primer | 0.1 | 10 | .1 | (1 uM) | 21 |
| (100 uM) | | | | | |
| RT-template diluted | 0.1 | 0 | 0 | (1:4000) | 21 |
| following pre-amp PCR | | | | | |
| dH20 | 2.8 | 0 | 0 | | 588 |
| total Volume | 10 | | | | 2100 |
| 95 C. 10 min, (95 C. 15 sec, | | | | | |
| 60 C. 1 min) × 40 | | | | | |

The present teachings further explored various cell lysis conditions effective for the isolation of micro RNA molecules. Generally, treatment at 95 C for 5 minutes was found to release micro RNAs from stem cells. Treatment for 99 C for 10 minutes was found to degrade about 50 percent of the micro RNAs released from the stem cells. Less than 1 percent of the micro RNAs were released from stem cells at 4 C. A variety of methods can be employed for isolating single cells prior to lysis, including for example aspiration with a micropipette tip, laser capture microdissection, fluorescent activated cell sorting of labeled cells (FACS), and other techniques readily available to one of ordinary skill in the art of molecular and cell biology.

In the context of stem cell identification, and of course other application areas, the present teachings contemplate embodiments in which small numbers of cells are analyzed, including a single cell. In some embodiments, the present teachings provide for analysis of one or more target micro RNA sequence molecules in a single cell. In some embodiments, the present teachings provide for analysis of one or more target micro RNA sequence molecules in five or fewer cells. In some embodiments, the present teachings provide for analysis of one or more target micro RNA sequence molecules in ten or fewer cells. In some embodiments, the present teachings provide for analysis of one or more target micro RNA sequence molecules in fifty or fewer cells. In some embodiments, the present teachings provide for analysis of one or more target micro RNA sequence molecules in one hundred and fifty or fewer cells. In some embodiments, the present teachings provide for analysis of one or more target micro RNA sequence molecules in greater than one hundred and fifty cells. As discussed supra, any of a variety of amplification strategies can be employed in the context of the present teachings for the analysis of small numbers of cells. The test samples from which such small numbers of cells can be recovered comprise conventionally fixed and stained histological and cytological preparations on microscope slides, single cells dissected from early-stage embryos generated by in vitro fertilization, microdissected needle-biopsy cores, blood samples, and forensics samples. Laser-capture microdissection is another attractive method of recovering diagnostic cells from histological preparations. Such laser-capture systems are commercially available from such sources as Arcturus (for example, the Veritas™ Microdissection Instrument).

Although the disclosed teachings have been described with reference to various applications, methods, kits, and compositions, it will be appreciated that various changes and modifications may be made without departing from the teachings herein and the claimed invention below. The foregoing examples are provided to better illustrate the disclosed teachings and are not intended to limit the scope of the teachings presented herein.

1. A method of forming a micro RNA signature from a small sample, said method comprising;
contacting a first target micro RNA with a first stem-loop primer, and a second target micro RNA with a second stem-loop primer;
reverse transcribing the first micro RNA and the second micro RNA by extension of the first stem-loop primer and the second stem-loop primer, to form a collection of extension products;
performing a PCR-based pre-amplification on the collection of extension products to form a collection of PCR-based pre-amplification products, wherein the collection of PCR-based pre-amplification products comprises a PCR-based pre-amplification first micro RNA product that was amplified with a first PCR-based pre-amplification primer pair, and a PCR-based pre-amplification second micro RNA product that was amplified with a second PCR-based pre-amplification primer pair;
dividing the collection of PCR-based pre-amplification products into a first vessel and a second vessel;
amplifying the PCR-based pre-amplification first micro RNA product in the first vessel with a first decoding primer pair, and amplifying the PCR-based pre-amplification second micro RNA product in the second vessel with a second decoding primer pair, wherein the first decoding primer pair comprises substantially the same sequence as the first PCR-based pre-amplification primer pair, and wherein the second decoding primer pair comprises substantially the same sequence as the second PCR-based pre-amplification primer pair;
detecting amplification of the first micro RNA target amplified in the first vessel and detecting amplification of the second micro RNA target amplified in the second vessel to form a micro RNA signature from the single cell; and,
forming a micro RNA signature from the single cell.

2. The method according to claim 1 wherein the reverse transcribing comprises cycling, and the cycling comprises 50-100 cycles of 18-22 C for 25-35 seconds, 40-45 C for 25-35 seconds, and 48-52 C for 1-2 seconds.

3. The method according to claim 1 wherein;
the first decoding primer pair comprises a universal reverse primer encoded by the first stem-loop primer, and a first micro RNA specific forward primer, wherein the first micro RNA-specific forward primer comprises a 3′ target-specific portion and a 5′ tail, and,
the second decoding primer pair comprises the universal reverse primer encoded by the second stem-loop primer, and a second micro RNA specific forward primer, wherein the second micro RNA-specific forward primer comprises a 3′ target-specific portion and a 5′ tail,
wherein the 5′ tail of the first micro RNA specific forward primer is the same sequence as the 5′ tail of the second micro RNA specific forward primer, wherein the universal reverse primer encoded by the first stem-loop primer is substantially the same sequence as the universal reverse primer encoded by the second stem-loop primer, and wherein the 3′ target specific portion of the first micro RNA specific forward primer is different from the 3′ target specific portion of the second micro RNA specific forward primer.

4. The method according to claim 1 wherein the small sample is a single cell.

5. The method according to claim 4 wherein the single cell is a putative stem cell.

6. A method of determining the identity of a single putative stem cell, said method comprising;
comparing a micro RNA signature for a single putative stem cell to a micro RNA signature from a known single stem cell; and,
determining the identity of the putative single stem cell.

7. The method according to claim 6 wherein the single putative stem cell is an embryonic stem cell, a neuronal stem cell, or a hematopoetic stem cell.

8. The method according to claim 6 wherein the putative single stem cell is a non-stem cell present in an in vivo stem-cell environment.

9. The method according to claim 6 wherein the putative single stem cell is a differentiated descendant cell derived from a precursor cell.

10. A method of determining the purity of a population of putative stem cells comprising;
providing an aliquot of the population of putative stem cells;
forming a micro RNA signature of the aliquot;
comparing the micro RNA signature of the aliquot to a micro RNA signature from a known cell; and,
determining the purity of the population of the putative stem cells.

11. The method according to claim 10 wherein the aliquot comprises embryonic stem cells, neuronal stem cells, hematopoetic stems cells, or combinations thereof.

12. The method according to claim 10 wherein the aliquot comprises a non-stem cell present in an in vivo stem-cell niche.

13. The method according to claim 10 wherein the aliquot comprises a differentiated descendant cell derived from a precursor cell.


SEQUENCE LISTING (3 sequences)

SEQ ID NO: 1 - DNA, 43 bp, Mus musculus
ctcaactggtgtcgtggagtcggcaattcagttgagcgccata

SEQ ID NO: 2 - DNA, 29 bp, Mus musculus
acactccagctgggtagcagcacgtaata

SEQ ID NO: 3 - DNA, 19 bp, Mus musculus
ttcagttgagccgccaata